(19) United States
(12) Patent Application Publication
(10) Pub. No.: US 2005/0065136 A1
(43) Pub. Date: Mar. 24, 2005

(54) Methods and compositions for the treatment of infertility using dilute hormone solutions

(76) Inventor: Russell R. Roby, Austin, TX (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 10/917,658

(22) Filed: Aug. 13, 2004

Related U.S. Application Data
(60) Provisional application No. 60/494,726, filed on Aug. 13, 2003.

(51) Int. Cl. A61K 31/57

(52) U.S. Cl. 514/170

(57) ABSTRACT

A method and composition for the treatment of infertility is disclosed. The method relates to using progesterone dilutions, or any other steroid hormone, to treat infertility. The hormone dilution may be administered sublingually, by drops or sublingual tablet or, an intradermal route of administration may be chosen.

### RELATED APPLICATION

This application claims priority to U.S. Provisional Patent Application Ser. No. 60/494,726, filed Aug. 13, 2003, and entitled “Methods and Compositions for the Treatment of Infertility Using Dilute Hormone Solutions.”

### TECHNICAL FIELD

The present invention relates in general to the treatment of infertility and in specific to the use of a dilute hormone solution for the treatment of infertility.

### BACKGROUND OF THE INVENTION

In 1995, it became clear that products of an activated immune system could play a role in infertility. Immune mediators could damage the placenta and cause miscarriages as well as damage the embryo and cause implantation failure. The immune cells that could be counted and assayed at that time were of a family called natural killer cells. These cells produce a cytokine called tumor necrosis factor alpha(TNF alpha). When this molecule is released and becomes higher than normal in the body it enters the embryo, the placental cells and even cells of other active organs in the body and causes DNA damage in the cell. The DNA of the rapidly dividing embryo cells is literally glued together and this stops cell division. The embryo or baby “withers of the vine” and dies.

There are now assays for intracellular cytokines including the T helper 1 (TH1) and T Helper 2 (TH2). These assays may reveal abnormal levels of TH1 and TH2 in infertile patients. There are also assays for anti-progesterone antibodies, the level or which may be abnormal in infertile patients.

While full-strength hormones have been used extensively in the therapy of infertility, there has been little investigation into the systemic manifestations or treatment of infertility as a condition of hormone allergy.

Hormone allergy has been previously described in the medical literature as premenstrual asthma. Skobeloff E. M., Spivey W. H., Silverman R. A., Ekin B. A., Harchelroad F. P., Alessi T. V.: The effect of the menstrual cycle on asthma presentations in the emergency department. Arch Intern Med 1996; 156: 1837-40. Claude F.: Asthma et menstruation. Presse Med 1938; 46: 755-759; Eliasson O., Scherzer H., DeGraff A. C.: Morbidity in asthma in relation to the menstrual cycle. J Allergy Clin Immunol 1986: 77: 87-94; Chandler M. H., Schuldheisz S., Phillips B., Muse K. N.: Pre-menstrual asthma: the effect of estrogen on symptoms, pulmonary function, and beta 2-receptors. Pharmacology 1997; 17(2): 224-234.

Premenstrual asthma is a condition where premenstrual fluctuations in hormones such as estrogen and progesterone cause the exacerbation of clinical symptoms. Exacerbations of symptoms appear to occur during the premenstrual period when progesterone levels are high. Several references have been made to a possible reaction to hormones. The first report of hormonal influence on asthma symptoms appeared in a case report by Frank from 1931. Frank, R T: The hormonal causes of pre-menstrual tension. Arch Neurol Psychiatry 1931; 26: 1053-57. Severe aggravation of asthma symptoms in one patient were clearly linked to oral contraceptives. Derimov, G S, Oppenheimer J: Exacerbation of premenstrual asthma caused by an oral contraceptive. Ann Allergy Asthma Immunol 1998; 81: 243-46.

Skobeloff et. al reported a four-fold increase in the presentation of asthmatic women to the emergency department during the perimenstrual interval (days 26 to 04 of the menstrual cycle). When Skobeloff's data is superimposed over the hormone levels during the menstrual cycle, it shows that the peak emergency room visits occurred during the premenstrual period when the progesterone is highest relative to estrogen (FIG. 1).

### SUMMARY OF THE INVENTION

Taken together, these data suggest, but do not indicate, that an allergic reaction to progesterone may be the cause of premenstrual asthma and, perhaps more broadly, other hormone allergy conditions. See e.g. U.S. Provisional Patent Application Nos. 60/591,638 filed Jul. 28, 2004 and 60/591,779 filed Jul. 28, 2004. These conditions may be treated with dilute solutions of the hormone allergen. According to the present invention, without being limited to any specific model or mode of action, infertility, particularly infertility attributable to hormone allergy, may be treated with dilute hormone solutions.

In accordance with teachings of the present invention, administration of dilute solutions of hormones may alleviate, ameliorate, or overcome infertility. The present invention provides methods and compositions useful in the treatment of infertility. According to some non-limiting embodiments of the invention, dilute solutions comprising progesterone and/or estrogen may be administered to a patient displaying symptoms of infertility. The invention also provides methods of treating infertility by administration of anti-IgE antibodies. The methods of the invention may be useful in both veterinary and human health applications.

### BRIEF DESCRIPTION OF THE DRAWINGS

A more complete and thorough understanding of the present invention and advantages thereof may be acquired by referring to the following description taken in conjunction with the accompanying drawings, in which like reference numbers indicate like features, and wherein:

FIG. 1 illustrates the number of female asthmatic patient visits to an emergency department during the menstrual cycle.

FIG. 2 illustrates the response of various hormone allergy symptoms to treatment with dilute hormone solution.

FIG. 3 illustrates responsiveness of asthma patients to dilute hormone solution treatment by percentage of asthma relief experienced.

### DETAILED DESCRIPTION OF THE INVENTION

The present invention relates to a treatment of infertility caused at least in part by a sensitivity or allergic reaction to hormones.

A method and composition for treatment of infertility using dilute hormone dilutions is provided. Observations that lead to and are a part of the present invention, suggest the possibility of an allergic reaction to the steroid hormone progesterone as a possible cause of infertility and other disorders.

One aspect of the present invention includes a previously unrecognized treatment for infertility that involves desensitizing a body's response to its own innate hormones. The treatment may be applied to any mammal including humans. In one embodiment, the mammal is a female with a clinical history of infertility.

While hormones may fluctuate throughout the menstrual cycle, treatment is not limited to any specific point in the menstrual cycle. In one embodiment, however, dilute solutions of progesterone are administered sublingually, every day or every other day, as needed, until there is an alleviation of a patient's clinical symptoms. These dilute formulations may be very similar to the type of dilutions that an allergist typically uses when treating allergic symptoms from external substances, or allergens, which are foreign to the body. However, in treating a patient with hormone allergy, instead of desensitizing the patient to a foreign substance, the patient is desensitized to his or her own innate hormone(s).

In accordance with some preferred embodiments of the present invention, dilutions of a hormone solution, such as progesterone, are used to treat infertility. A hormone dilution ranging in concentration from 5 mg/ml to 0.5 μg/ml is administered sublingually. Although a 10% solution is preferable for some patients, the strength of the dilution selected for treatment may be based on the severity of the patient's symptoms and prior treatment history. The amount, frequency and strength of the hormone dilution may be varied depending on severity of symptoms and on response achieved. The dilution may be in the form of a liquid solution that may be a suspension or drops or the dilution may be in the form of a sublingual tablet or any other oral formulation, liquid or solid, suitable for administration of hormone dilutions.

In an alternative embodiment of the invention, the route of administration may be intradermal. In accordance with a further aspect of this invention a dilute progesterone solution (concentration 5 mg/ml to 0.5 μg/ml) or a dilute estrogen solution (concentration 5 mg/ml to 0.5 μg/ml) may be administered to treat hormone allergy symptoms in females. A solution ranging from approximately a 1% dilution to a 20% dilution may be used or any other dilution suitable for achieving the desired clinical effect.

A composition of the present invention may include a standard solution of aqueous progesterone, or any other indicated steroid hormone, diluted with normal saline to achieve concentrations of a desirable concentration. The strength of a dilution selected for treatment may be based on severity of the patient's symptoms and prior treatment history. This selection methodology may be similar to that used in treatments with foreign allergens and appropriate selections for an individual patient will be apparent to one skilled in the art.

In one embodiment of the invention, 0.1 cc or a comparable sublingual tablet formed of a 10% dilution of progesterone is administered sublingually every day for sixty days. The frequency of administration may be increased or decreased as required, to achieve a desired treatment response. The strength of the hormone dilution selected for treatment may also be varied depending on severity of symptoms and on response achieved.

Before dilute hormone therapy is administered, baseline levels of serum progesterone antibodies may be measured. Response to therapy is measured by serum progesterone antibodies that may be assayed at any point during or after therapy. Response to therapy is also measured by pregnancy success rate. Intracellular ytokine assays may also be performed pre- and post-therapy to measure response rates to therapy.

In an alternative embodiment of this invention, the dilution may be administered intradermally for instance, in patients who may have no response to sublingual drops or patients who are unable to use the sublingual delivery method. Regardless of the route of delivery, compositions of the invention may be administered once or more than once. If administered more than once, the dosage may be the same or different each time. In some embodiments of the invention, the dosage may be increased at each administration. In other embodiments, the dosage may be decreased at each administration. In a non-limiting embodiment of the invention, the dosage of progesterone administered during the first week is 10⁻¹⁵, during the second week is 10⁻⁴, and during the third week is 10⁻³.

Infertility according to the invention may be any impediment on producing live off-spring. This includes, for example, any impairment of gamete formation, fertilization, gestation or delivery. Non-limiting examples include defects in spermatogenesis, oogenesis, ovulation, embryogenesis, implantation, in utero development, and cervical dilation.

Infertility attributable to hormone allergy may be accompanied by other hormone allergy-induced symptoms, which may include irritation or pain in the neck, nose, eyes, throat, ear, skin, back, hips, upper extremities and lower extremities, headache, including migraine headaches, shortness of breath, including asthma, arthritis, fibromyalgia, chronic fatigue syndrome and multiple sclerosis. In general, when the hormone allergy includes an allergy to progesterone or estrogen, these symptoms will be treated in female patients.

Certain embodiments of the present invention are related to methods of diagnosing hormone allergy in patients, particularly female patients. Because the presence of IgE is required for a Type 1 allergic reaction, detection of elevated of anti-hormone IgE is indicative of a Type 1 hormone allergy. Presence of IgG, IgM or IgA may be indicative of a Type 2 or 3 hormone allergy. An assay for such Igs may be particularly useful in patients exhibiting the symptoms or disorders described above. Detection of elevated anti-hormone Ig provides a clue as to which hormone may be responsible for the symptoms or disorder, thus guiding treatment. Failure to detect elevated levels of anti-hormone Ig may indicate that the symptoms or disorder are caused by something other than hormone allergy, such as a different autoimmune disorder.

The present invention also relates to a method of treating infertility attributable to IgE-mediated hormone allergy by administration of an anti-IgE treatment. The anti-IgE treatment may be any treatment sufficient to reduce or inhibit IgE levels in a manner to alleviate symptoms of the infertility. In more specific embodiments, the treatment may include an anti-IgE antibody, such as a humanized antibody. For example, it may include the antibodies currently produced by Genentech, Calif. and marketed as Xolair, or by Tanox, Tex. currently labeled as TNX-901.

Other anti-IgE antibodies may be developed and testing using methods known to the art to determine if they are more effective against hormone allergy or in the relief of any particular disease or symptom, such as one of those described above.

The anti-IgE antibodies may be administered in any appropriate manner or formulation. In specific embodiments, they may be administered by injection in a pharmaceutically acceptable carrier, such as a saline-based carrier.

The anti-IgE antibodies may be administered continuously or intermittently, but in specific embodiments, they may be administered at the onset of symptoms, or at another time-appropriate juncture, such as before conception is desired in infertile patients.

### EXAMPLES

### Example 1

### Dilution Protocol

Progesterone USP 50 mg/ml (Schein Laboratories, Florham, N.J.) is diluted with physiologically-compatible(normal) saline to produce the progesterone dilutions used in treatments. The initial progesterone is suspended in sesame oil. Therefore, to achieve an even suspension, the vial must be vigorously shaken at each stage of the initial preparation and before use of each vial. The first dilution is made by adding 0.5 ml of progesterone to 4.5 ml normal saline. This results in a 1:10 dilution of progesterone (progesterone 5 mg/ml) which is labeled “PROG 1.” After vigorously shaking the PROG 1 vial, 0.5 ml is withdrawn and injected into the next vial of 4.5 ml of normal saline. This results in a 1:100 dilution of Progesterone (0.5 mg/ml, “PROG 2”). To produce the next dilution, an aliquot of 0.5 ml is immediately withdrawn from a vial of PROG 2 and injected into the next vial of 4.5 ml of normal saline. This results in a 1:1000 dilution of Progesterone (50 μg/ml “PROG 3”). These steps are repeated until there are five serial dilutions labeled “PROG 1” through “PROG 5.” (See Table 1). A milligram (mg) is defined as 1/1000 or 10-3 of a gram. A microgram (μg) is defined as 1/1,000,000 or 10-6 of a gram.

TABLE 1
Progesterone Dilutions

| | Progesterone | | Dosage Used |
|---|---|---|---|
| Label | Concentration | Dilution | (0.1 ml) |
| PROG 1 | 5 mg/ml | 10-1 | .5 mg |
| PROG 2 | .5 mg/ml | 10-2 | .05 mg |
| PROG 3 | 50 μg/ml | 10-3 | 5 μg |
| PROG 4 | 5 μg/ml | 10-4 | .5 μg |
| PROG 5 | .5 μg/ml | 10-5 | .05 μg |

### Example 2

### Anti-Hormone Antibodies in Patients Responding to Hormone Allergy Treatment

Patients undergoing treatment for hormone allergy involving intradermal or sublingual administration of dilute progesterone were tested by prick test for the presence of anti-progesterone IgG, IgM, or IgE antibodies using an ELISA assay. Blood samples obtained from 271 unselected patients were tested for anti-progesterone IgG and IgM. In these patients, 52% exhibited high IgG or IgM antibodies. Blood samples from 88 different patients and 88 healthy individuals were tested for anti-progesterone IgE and anti-estrogen IgE. Anti-progesterone IgE antibodies were high in 41% of patients tested. Anti-estrogen IgE antibodies were high in 86% of patients tested.

Over half of all patients who reported relief of at least 60% of the twelve hormone allergy symptoms described further in Example 2 when administered dilute progesterone additionally had high levels of anti-progesterone IgG, IgM and IgE.

This indicates that in a significant number of patients may have a Type 1 allergic reaction to progesterone. This type of reaction is very rapid and often quite dangerous, even when compared to other types of allergic reactions. Because it is so rapid, it does not require long-term exposure to an antigen or exposure to a large amount of antigen.

Additionally, the presence of elevated IgG, and IgM in hormone allergy patients indicated that some responses may also be Type 2 or 3 allergic responses.

Finally, the absence of anti-progesterone antibodies in several hormone allergy patients may indicate a Type 4 allergic response.

### Example 3

### Treatment of Hormone Allergy Symptoms

Patients experiencing several hormone allergy symptoms experienced relief when administered dilute doses of progesterone either sublingually or intradermally. Further, as compared to several other antigens, progesterone proved most effective in producing these effects. Additionally, relief is experienced very quickly in most patients, further suggesting that their progesterone allergy might be a Type 1 allergic reaction.

The symptoms measured were irritation or pain in the neck, nose, eyes, throat, ear, skin, back, hips, upper extremities and lower extremities, headache, and shortness of breath. The patients were administered 0.5 mg of progesterone intradermally.

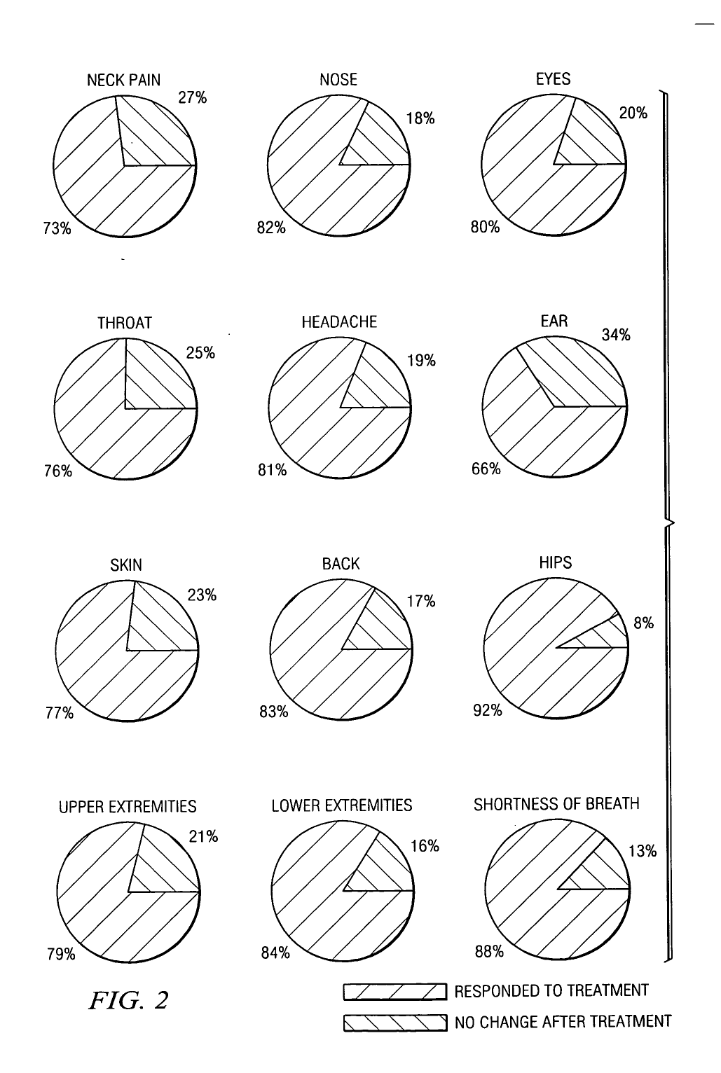

The results of these tests are summarized in Tables 2 and 3 and FIG. 2.

TABLE 2
Clinical Responses to Intradermal
Administration of Progesterone

| | | | % of Patients | % Improvement |
|---|---|---|---|---|
| | # of | # of Responding | who | in Responding |
| | Patients | Patients | Respond | Patients |
| Neck | 144 | 104 | 72% | 62% |
| Nose | 109 | 89 | 82% | 63% |
| Eyes | 90 | 73 | 81% | 57% |
| Throat | 92 | 70 | 76% | 64% |
| Headache | 103 | 84 | 82% | 69% |
| Ear | 35 | 23 | 66% | 25% |
| Skin | 34 | 27 | 79% | 74% |
| Back | 57 | 47 | 82% | 72% |
| Hips | 26 | 24 | 92% | 79% |
| Lower | 61 | 48 | 79% | 76% |
| Extremity | | | | |
| Upper | 50 | 40 | 80% | 75% |
| Extremity | | | | |
| Shortness | 142 | 124 | 87% | 67% |
| of Breath | | | | |

Patients were designated as responding patients in Table 2 if they experienced any relief of the indicated symptom. Patients were also asked to estimate the amount of improvement in the symptom, an average of which is indicated as the % improvement in responding patients.

TABLE 3
Probability of Results due to Random
Effects

| | | | | Probability |
|---|---|---|---|---|
| | | | | of + Results |
| | # of Patients | | # of non- | due |
| | with | # of Responding | Responding | to Random |
| | Symptom | Patients | Patients | Effects |
| Neck | 146 | 106 | 40 | <=0.0001% |
| Nose | 112 | 92 | 20 | <=0.0001% |
| Eyes | 94 | 75 | 19 | <=0.0001% |
| Throat | 94 | 71 | 23 | <=0.0001% |
| Headache | 105 | 85 | 20 | <=0.0001% |
| Ear | 35 | 23 | 12 | <=5% |
| Skin | 35 | 27 | 8 | <=1% |
| Back | 58 | 48 | 10 | <=0.0001% |
| Hips | 26 | 24 | 2 | <=0.0001% |
| Lower | 51 | 43 | 8 | <=0.0001% |
| Extremity | | | | |
| Upper | 63 | 50 | 13 | <=0.0001% |
| Extremity | | | | |
| Shortness | 144 | 126 | 18 | <=0.0001% |
| of Breath | | | | |

In Table 3 the probability that positive results (i.e. patients responding to treatment) were due to random effects was calculated using standard statistical analysis of patient tests. It was assumed that if the treatment has no effect, there is a 50% chance that a patient will indicate a positive response and a 50% chance that a patient will indicate a negative response. From this assumption, coupled with the actual data, one may calculate the probability that positive responses are due to an actual effect of the treatment and not simply random effects. In general, results with a less than 1% chance of being random are considered highly significant. Results with a less than 5% chance of being random are normally conclusively labeled as nonrandom. Thus, the results observed for most of the symptoms in Table 3 are highly significant, with only the results for ear irritation and pain falling merely in the nonrandom category.

The results of Table 3 are depicted graphically in FIG. 2.

Further tests have shown that many patients whose symptoms are not relieved using progesterone treatment do experience relief when administered 0.4 mg of estrogen intradermally.

### Example 4

### Hormone Allergy in Female Asthmatics

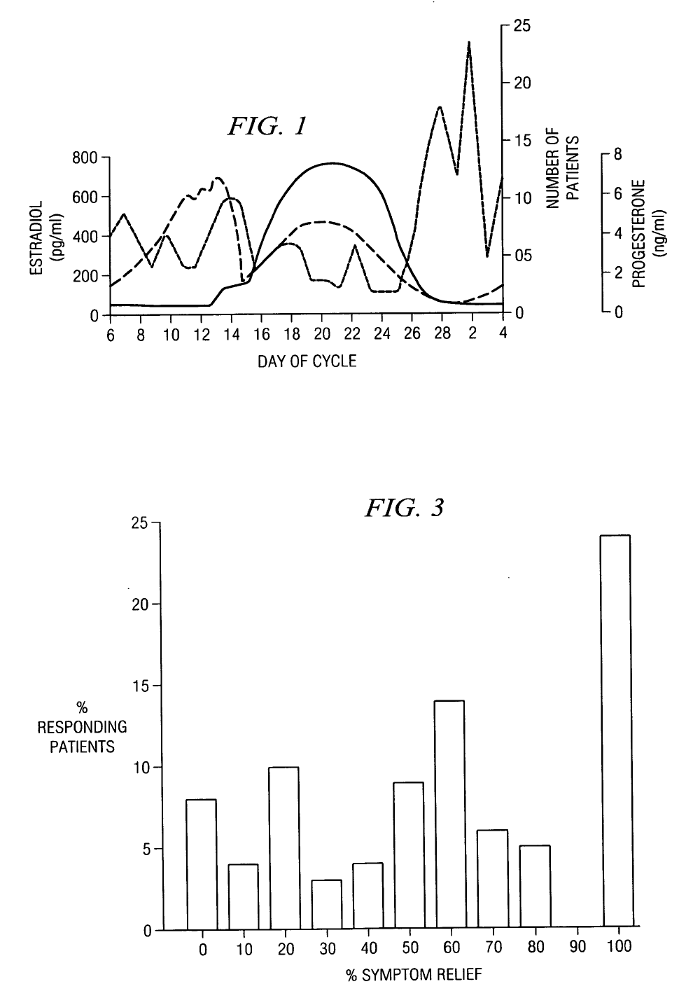

FIG. 1 shows that hospital admissions for asthma symptoms are highest during the perimenstrual interval (days 26 to 04) of the menstrual cycle when progesterone levels are highest relative to estrogen levels. Based on the results presented in FIGS. 1 and 2, particularly those related to relief of shortness of breath, coupled with this increase in hospital admissions, it appears that there may be a relationship between premenstrual asthma and hormone allergy.

To test this hypothesis, 142 women with acute asthma were asked to evaluate the severity of their symptoms using an eleven point scale. Ten was assigned to the most severe symptoms, while 0 was assigned to an absence of symptoms. Immediately after assessing their symptoms on this scale, patents were administered 0.5 mg of progesterone by intradermal injection.

Approximately 15 to 30 seconds after the injection was administered, each patient was asked if her symptoms were worse, better, or unchanged. Each patient was also asked to rate her present symptoms on the same 0 to 10 scale.

The results of these tests are shown in FIG. 3. FIG. 3 shows the % of patients responding to treatment by % of symptom relief reported. Of the 142 patients tested 129 (91%) positively responded to the treatment. Using the random effects calculations described in Example 3, this correlates to a less than 0.1% chance that the results are random. Thus the results may be considered highly significant.

Of the 129 patients who experienced symptom relief, 40 (28%) reported 100% relief of their asthma symptoms. Using the 0 to 10 scale described above, the average symptomatic relief for all patients (those responding and those not responding to treatment) was 3.4 points. The average symptomatic relief for those who reported some response to the treatment was 5 points. Response change relative to the maximum individual patient score averaged a 62% reduction in symptoms.

### Example 5

### Double-Blind, Randomized, Placebo-Controlled Study of Hormone Allergy Treatment in Adult Female Patients with Asthma, Pain, Migraine Headaches, Infertility, Fibromyalgia, Chronic Fatigue Syndrome, or Multiple Sclerosis

As described above in Examples 2 to 4, a number of patients with various symptoms exhibit anti-progesterone or anti-estrogen antibodies that might indicate allergies to these hormones. In addition, many of these patients show a rapid reduction in symptoms in response to administration of small doses of progesterone or estrogen sublingually or intradermally (doses are normally 0.5 mg or 0.4 mg, respectively).

During the studies described in Examples 3 and 4, it became apparent that the hormone allergy treatment the patients were receiving might also treat other symptoms or disorders experienced by these patients. Specifically, patients with arthritis, asthma, pain, migraine headaches, fibromyalgia, chronic fatigue syndrome and multiple sclerosis showed a reduction of disorder-related symptoms by at least 60% in over half of all patients tested. Further, of 16 infertile patients receiving hormone allergy treatment, two became pregnant and one delivered a healthy baby.

Additionally, anti-hormone antibodies were found in several multiple sclerosis patients whose multiple sclerosis-related symptoms also improved during hormone allergy treatment. Although multiple sclerosis is directly mediated by anti-myelin hormones, it appears that the hypersensitivity reaction involved in hormone allergy may aggravate the disorder. Given that various mediators of the immune response are known to be shared between the immune reaction giving rise to multiple sclerosis and the immune reaction giving rise to allergy, this aggravation of multiple sclerosis symptoms by hormone allergy appears plausible. It is possible that shared components of the immune reaction may also explain some of the positive results seen when hormone allergy is treated in patients with other disorders mediated by the immune system, such as arthritis and asthma and also possibly infertility.

The beneficial effects of hormone allergy treatment through administration of dilute amounts of progesterone or estrogen may be further confirmed in a double-blind clinical trial.

Potential subjects may be asked to rate their symptoms on a scale of 0 to 10, with 0 representing no symptoms and 10 representing the worse symptoms the patient experiences. Subjects rating their symptoms at level 6 or higher may be selected for the study. Patients selected may then be administered 0.5 mg of progesterone sublingually. If the patient indicates a decrease in symptom level on the 0 to 10 scale by at least 50% within 60 seconds, the patient may be administered progesterone during the study. If not, the patient may be similarly tested with 0.4 mg of estrogen administered sublingually. If the patient responds to the estrogen, then the patient may receive estrogen during the study.

Patients who qualify under the initial progesterone or estrogen tests may then be tested to see if they have high levels of anti-progesterone and/or anti-estrogen IgE. Only those exhibiting high levels of at least one of these two antibodies may be included in the trial.

Next, patients may be asked whether they have a history of regularly experiencing symptoms higher than 5 on the scale of 0 to 10. Those who do not may be excluded from the trial.

Other relevant assessments of the patients' disorder severity and symptoms may also be made at the beginning of the trial, before hormone treatment.

Patients enrolled in the trial may then receive sublingual doses of either 0.5 mg of progesterone or 0.4 mg of estrogen twice daily for 90 days. Control subjects may receive a placebo containing no hormone. Enough patients may be enrolled for each disorder group to give statistically significant results. Similarly, enough patients may be included in the control groups for each disorder group to give statistically significant results. For example, 100 subjects may be enrolled in the trial for each of the eight disorders. Of these subjects, 90% may be assigned to the test group and 10% may be assigned to the control group.

Presence of the relevant disorder may be determined based on a previous diagnosis by a physician. Infertility may be defined as having undergone one or more failed in vitro fertilization attempts or two or more spontaneous abortions. Patients may be females between the ages of 20 and 70.

At the end of the 90 days, patient anti-hormone IgE levels may be retested. Disorder symptoms may also be reassessed. Usage of other medications to treat the patients' symptoms or disorders may also be monitored throughout or at the end of the study to determine if the hormone treatments cause any alterations in the patients' usage of other medications.

Standard statistical methods may be used to analyze study results, such as changes in anti-hormone IgE levels, symptom ratings, symptoms and medication usage.

1. A method for treating infertility comprising administering to a human an effective amount of a hormone dilution.

2. The method of claim 1 wherein the hormone dilution comprises a steroid hormone solution with a concentration between approximately 5 mg/ml and 0.5 μg/ml.

3. The method of claim 1, wherein the hormone dilution comprises progesterone.

4. The method of claim 1 further comprising the concentration of the hormone solution selected from the group consisting of 5 mg/ml, 0.5 mg/ml, 50 μg/ml, 5 μg/ml, and 0.5 μg/ml.

5. The method of claim 1 wherein the hormone dilution comprises a progesterone dilution of 10%.

6. The method of claim 6 further comprising the amount of progesterone administered ranges from between approximately 0.5 mg to 0.05 μg per dose.

7. The method of claim 1 wherein the hormone dilution is administered sublingually.

8. The method of claim 1 wherein the hormone dilution is administered in a sublingual tablet.

9. The method of claim 1 wherein the hormone dilution is administered in sublingual drops.

10. A method of treating infertility with a hormone dilution comprising:
administering a dilution of progesterone, the dilution configured to be administered sublingually; and
administering additional dilutions of progesterone as often as necessary to stimulate an effective response.

11. The method of claim 10 wherein the hormone dilution comprises drops.

12. The method of claim 10 wherein the hormone dilution comprises a sublingual tablet.

13. A method of treating infertility using sublingual progesterone dilutions comprising:
administering the progesterone dilution once a day, the progesterone dilution comprising a sublingual tablet or solution formed of 10% progesterone.

14. The method of claim 13, wherein the daily progesterone dilution administration continues for approximately sixty days.

15. The method of claim 13 further comprising measuring serum anti-progesterone antibodies.

16. The method of claim 13 further comprising measuring intracellular cytokines.

17. A method for treating infertility comprising administering to a human an effective amount of a humanized anti-IgE antibody.
